(19) United States
(12) Patent Application Publication
(10) Pub. No.: US 2012/0083648 A1
(43) Pub. Date: Apr. 5, 2012

(54) Apparatus and Method for Relieving Stress

(75) Inventors: Catherine D. Dowdell, Caledonia, WI (US); John R. Weaver, Waukesha, WI (US); Kristina J. Genslak, Racine, WI (US); James R. Crapser, Racine, WI (US)

(73) Assignee: S.C. JOHNSON & SON, INC., Racine (US)

(21) Appl. No.: 12/897,461

(22) Filed: Oct. 4, 2010

(51) Int. Cl. A61M 21/00

(52) U.S. Cl. 600/26

(57) ABSTRACT

A device for relieving user stress and method of using same are disclosed. The device may be provided in the form of a kit that the user may use to relieve himself or herself of troubling thoughts and use the dissolution features of the kit to visually observe the thought melting away. The kit may include a plurality of dissolvable substrates on which thoughts can be recorded, a writing instrument to record the thoughts on the substrate, a vessel to contain a solvent, and a medical device to monitor one or more physiological parameters of the user. The physiological parameters of the user may include blood pressure, heart rate, respiration rate, brain function, and combinations thereof.

### FIELD OF THE DISCLOSURE

The present disclosure generally relates to stress-relieving devices and methods, and more particularly relates to stress-relieving devices and methods which cause a physiological shift from the predominant use of the sympathetic nervous system to the predominate use of the parasympathetic nervous system.

### BACKGROUND OF THE DISCLOSURE

Studies show that modern society is the most stressed population ever in existence. People often work longer now for less pay, try to balance a healthy home life with work life, raise their children, eat right, exercise, stay active in their communities, tend to their elders, and on and on. This is at the same time the world at large seems to be in constant turmoil, be it with regard to wars abroad, poverty at home, political distrust, crime, or societal expectations.

With these factors in play, the market for stress relieving agents or technologies has never been in more demand. The pharmaceutical industry has exploded in the past few decades, in part, due to these forces. Such pharmaceuticals can be as straightforward as mild sedatives or sleeping pills purchased over to the counter, to stronger depressants or insomnia aids able to ensure a good night's sleep. Anti-anxiety medications are also quite prevalent and regularly prescribed by the medical community. Even self-medicated drugs such as alcohol and tobacco are often used to alleviate stress.

While each is arguably effective in its own way, the offerings of the medical, pharmaceutical and consumer products industries are not without their drawbacks. Some are expensive, others are addictive, some are fattening, others are socially unaccepted. With this in mind, many people have traditionally or alternatively turned to holistic or organic or physical therapy approaches to relieve their stress levels. Such approaches include yoga, meditation, and massage. Herbal teas, aromatherapy, acupuncture, and consumption of organic foods are other examples.

One problem associated with many of these approaches is that their benefits are often difficult or impossible to quantify. What might work for one person might be wholly ineffective on another person. Still others simply do not work, but persist due to public perception or traditionally held beliefs.

With this in mind, the market continues to need an alternative stress-relieving apparatus or method that can relieve stress without medical consequences, addictions or large expense, and which can be verified, relied upon, and clinically proven.

### SUMMARY OF THE DISCLOSURE

In accordance with one aspect of the disclosure, a kit adapted to relieve stress is disclosed which comprises a supply of dissolvable substrates, a vessel adapted to hold a supply of solvent, and a writing instrument adapted to write on the supply of substrates.

In accordance with another aspect of the disclosure, a method of improving at least one physiological parameter of a user is disclosed which comprises recording information on a tangible substrate, dissolving the substrate in a solvent, and allowing the user to observe the substrate transforming from a solid to a liquid for a timed duration sufficient to alter the at least one physiological parameter.

In accordance with a still further aspect of the disclosure, a kit adapted to relieve user stress is disclosed which comprises a dissolvable recording media and a vessel adapted to hold a supply of solvent.

These and other aspects and features of the present disclosure will be better understood upon reading the following detailed description when taken in conjunction with the accompanying drawings.

### BRIEF DESCRIPTION OF THE DRAWINGS

FIG. 1 is a schematic representation of a stress-relieving apparatus constructed in accordance with the teachings of the present disclosure;

FIG. 2 is a side view of a stress-relieving apparatus constructed in accordance with the teachings of the disclosure;

FIG. 3 is a cross-sectional view of the apparatus of FIG. 2 taken along line 3-3 of FIG. 2; and

FIG. 4 is a flowchart depicting a sample sequence of steps which may be practiced in accordance with the teachings of the present disclosure.

While the present disclosure is susceptible to various modifications and alternative constructions, certain illustrative embodiments thereof have been shown in the drawings and will be described below in detail. It should be understood, however, that there is no intention to limit the present invention to the specific forms disclosed, but on the contrary, the intention is to cover all modifications, alternative constructions, and equivalents falling within the spirit and scope of the present disclosure.

### DETAILED DESCRIPTION

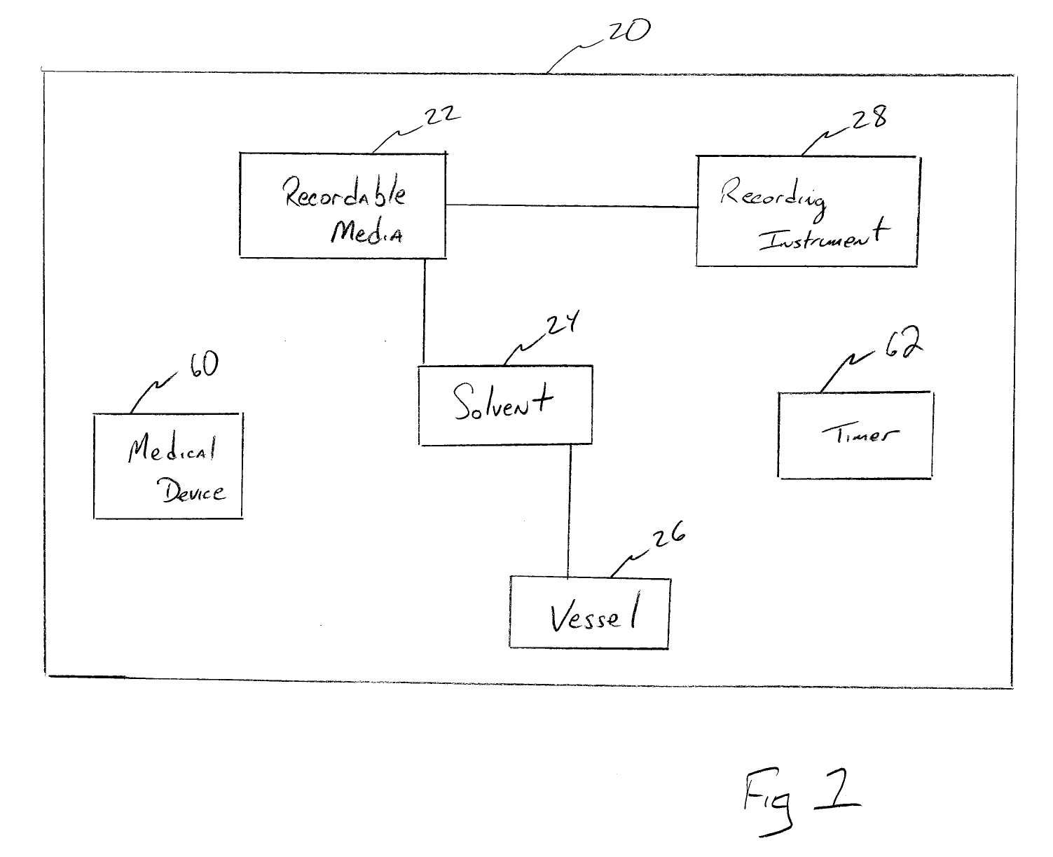

Referring now to the drawings, and with specific reference to FIG. 1, an apparatus for relieving user stress is generally referred to by reference numeral 20. The apparatus may be sold as a kit to be assembled and employed by a user, or may be sold as separate constituents individually assembled. Moreover, while broadly disclosed with respect to a written media and solvent, it is to be understood this is but one embodiment to be disclosed herein, and that other embodiments employing the teachings of the present disclosure are certainly possible.

Beginning with FIG. 1, the kit 20 is shown to include a recordable media 22. Such recordable media 22 could be provided in the form of a supply of substrates adapted to be written upon such as paper, polymeric sheets, soap, cellulosics or the like. For example, in one embodiment, the recordable media may be dissolvable planar sheets, blocks, scrolls or other shapes manufactured from a surfactant-based media soluble in water. Sheets made of dehydrated soap may be particularly effective. In other embodiments, the recordable media may be provided in the from of paper adapted to be combusted with fire, a polymeric sheet adapted to dissolved in a petroleum-based solvent or melted upon exposure to heat, a electronic media adapted to magnetically record or digitally record audio or video messages to be destroyed upon exposure to electromagnetic fields, heat, solvents, or mechanical destruction.

With respect to solvents, FIG. 1 also depicts the kit 20 may include a supply of solvent 24. In the example of a surfactant-based media 22, the solvent could simply be water or a water-based solvent. In alternative embodiments, depending upon the media being used, the solvent could be petroleum-based. In still further embodiments, the solvent could be replaced by an acid or base, a heat source, an electromagnetic field or the like. In a still further embodiment, the kit 20 may not include any supply of solvent, but rather the user could simply provide the solvent, such as warm, or any other temperature, water from a tap.

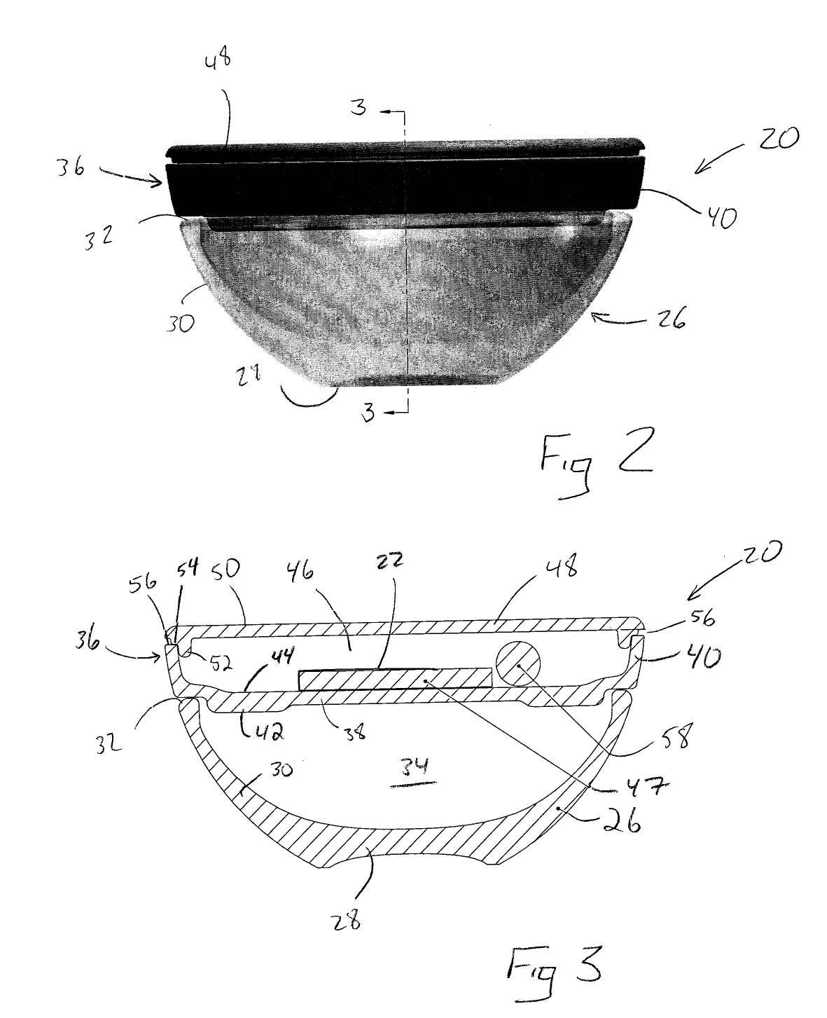

The kit 20 may further include a vessel 26 to hold the solvent 24 during use. As shown in FIGS. 2 and 3, the vessel 26 may be substantially bowl-like in shape including a bottom wall 28, from which upwardly flares a side wall 30 terminating in an upper rim 32. The vessel 26 defines an internal cavity 34 into which the solvent can be poured.

In order to temporarily close the vessel 26, such as prior to sale, during shipping, or after purchase and while not in use, a lid assembly 36 may be provided. The lid assembly 36 may include a primary lid 38 sized to rest directly atop the rim 32. The primarily lid 38 may further include an upwardly extending circumferential ridge 40, as well as a downwardly extending circumferential detent 42. The ridge 40 may extend sufficiently above a plate 44 of the primary lid 38 so as to define an internal recess or opening 46 for storage of the supply of dissolvable substrates 22. More specifically, the substrates 22 are shown stacked within a carton 47, made of paper or the like, and adapted to slide out one at a time upon grasping by the user. The detent 42 is sized and positioned to provide centering of the lid 38 on the vessel 26, while also providing a positive stop hindering inadvertent sliding of the lid 38 off the vessel 26.

The lid assembly 36 may further include a secondary lid 48 adapted to sit atop the primary lid 38. The secondary lid 48 may include a planar top surface 50 with a downwardly depending circumferential rib 52. The rib 52 is sized and positioned to center the secondary lid 48 on the primary lid 38. Moreover, the rib 52 may include a radially extending shoulder 54 to contact the top 56 of the ridge 40 and in so doing hold the planar top surface 50 away from the rim 32 a sufficient reveal distance 56 to facilitate grasping and removing same by a user. While not depicted the planar top surface 50 may include decorative graphics, textures or the like.

The kit may further include a recording instrument 58. As shown in FIG. 3, the recording instrument 58 is sized so as to be storable in the recess 46. In the example of a surfactant-based substrate, the recording instrument may be a writing instrument such as a pen, marker, pencil, or other writing instrument compatible with the medium. The ink may be water-based as well to work with the surfactant of the sheets 22, and be provided with a formulation to “lay-down” on such a slippery surface. In a similar regard, it is important to also note that the pen 58 should not have a blunt enough tip to tear the sheet 22 when writing. In other surfactant-based embodiments, depending on the relative dimensions of the medium, the recording instrument may be provided in the form of a knife, awl, or other utensil adapted to carve messages into the medium. In the example of electronic media, the recording instrument 58 may be provided in the form of an audio or video recorder including such technologies as digital video discs, compact discs, flash drives, magnetic tapes, and the like.

As the apparatus and method of the present disclosure is concerned with relieving stress and quantifying same, the kit 20 may also include a medical device 60. The medical device 60 may be any type of medical device able to track and monitor at least one physiological parameter of the human body including, but not limited to, blood pressure, heart rate, respiratory rate, brain activity, and various combinations thereof. Moreover, as the kit 20 is adapted to be used for a set period of time in order not only to alter one or more of such physiological parameters, but also to facilitate a shift of the user from the predominate use of the sympathetic nervous system to the predominate use of the parasympathetic nervous system, a timer 62 may also be included to ensure the kit 20 is used for a sufficient long duration.

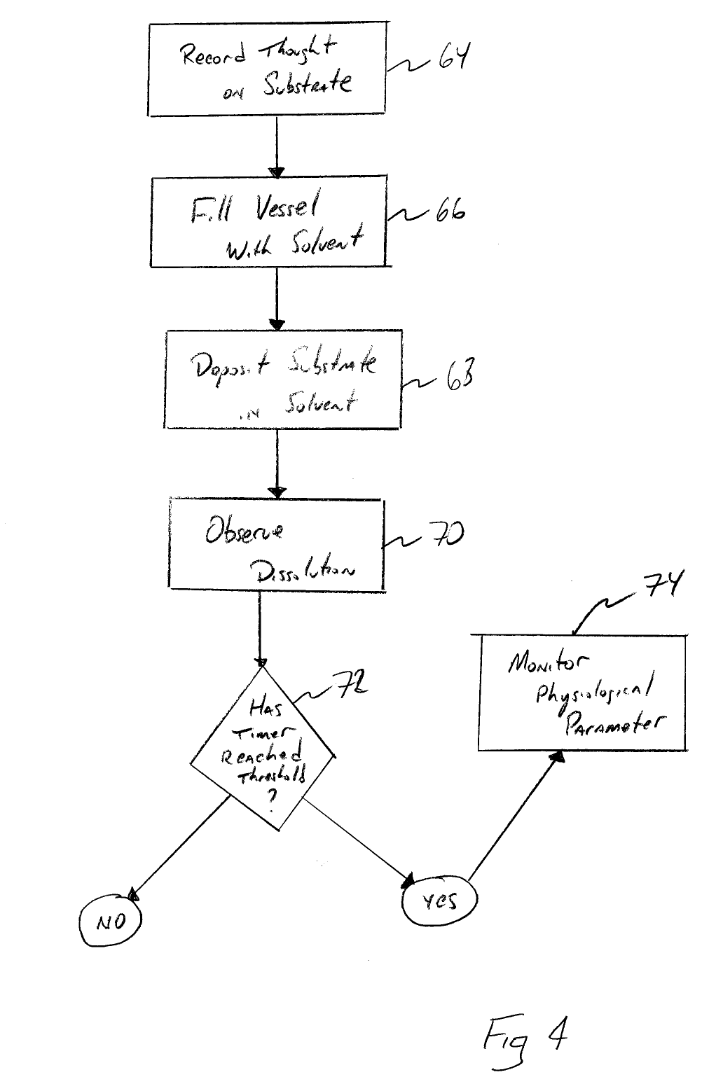

Turning now to FIG. 4, the method in which the kit 20 may be used is set forth by listing a sample sequence of steps which may be practiced in accordance with its teachings. It is to be understood that such steps and the specific sequence of steps are provided by way of example only and in no way should be found to limit the scope of the disclosure.

In order to most effectively navigate a user's central nervous system from the predominate use of the sympathetic to the parasympathetic, the inventors have found it advantageous to have a user record his or her troubling or stress-inducing thoughts in a given media such as the aforementioned surfactant-based substrates, polymeric sheets, paper, or electronic media. This is set forth as step 64 in FIG. 4. By way of background, it is important to note that the “sympathetic nervous system” as used herein refers to the state of the brain and nervous system when most alert, acting voluntarily or reacting to a stressful situation, whereas “parasympathetic nervous system” as used herein refers to the state of the brain and nervous system when acting automatically, or when at rest. For example, the parasympathetic nervous system governs involuntary acts such as but not limited to breathing, digestion and salivation. The inventors have found that it is the transformation of the nervous system from the sympathetic to the parasympathetic that allows for the greatest stress relief and that is achievable by the teachings of the present disclosure.

Once recorded, the user may immediately feel a sense of relief simply by expressing those thoughts and seeing them in tangible form. However, to even more effectively relieve that stress, the inventors have found it also advantageous to have the user dissolve, disintegrate, or otherwise destroy the recorded media. The act of such dissolution or other destruction facilitates the user in letting go of the given thought and in turn relieving himself or herself of the stress associated with that thought.

In terms of the process steps of FIG. 4, this may involve having the user fill the vessel 26 with the solvent 24 as shown by a step 66 and then deposit the substrate 22 into the solvent 24 as shown by a step 68. Alternatively, the substrate can first be inserted into the vessel 26 and the solvent 24 can then be added. In still further embodiments, the media may be burned or exposed to a heat source to effect melting of the substrate 22. With electronic media, an electromagnetic field may be generated such that upon sufficient exposure of the media to the field, the message may be erased. Similarly, the electronic media by be exposed to a solvent, acid or heat source to cause destruction of same.

Upon exposing the recorded media 22 to the solvent 24, a next step in the process is for the user to observe the dissolution of the media 22 as shown by a step 70 in FIG. 4. The inventors have found that it this act of observation, or visually inspecting the breakdown, melting, dissolution or other form of destruction of the thought captured on the media relieves the stress of the user and manifests itself in the form of a lowered physiological parameter such as blood pressure, heart rate, respiratory rate, brain activity, or the like and facilitates movement of brain activity from the sympathetic to the parasympathetic.

In order to most effectively ensure this transformation, the inventors have found it beneficial for a user to observe the dissolution for a set period time. This duration or threshold time limit is reflected in step 72 of FIG. 4. For example, the inventors have found a minimum threshold of five minutes is likely to result in quantifiable reductions in the aforementioned physiological parameters. Accordingly, in FIG. 4, it will be noted that if the threshold question 72 is answered in the negative, the user is directed to continue observation of the dissolution, whereas if the threshold question 72 is answered in the affirmative, the user may choose to cease observation.

As an optional further step, the user may then opt to monitor at least one physiological parameter as shown by a step 74. With this in mind, the kit 22 may include the aforementioned medical device 60 to facilitate this monitoring. For example, the medical device 60 may be a blood pressure cuff, a heart rate monitor, a thermometer, a pulse-oxygen monitor, or a combined tool adapted to measure one or more physiological parameters. In so doing, the user may be provided with quantified data reflecting his or her stress relief in addition to his or her general sense or well being.

From the foregoing, it can be seen that the present disclosure sets forth an apparatus and method that a user can use to relieve stress. The apparatus may include a vessel adapted to hold a supply of solvent, as well as a lid assembly adapted to store a supply of dissolvable or otherwise destructible substrates and a recording instrument. By recording troubling thoughts or other sources of stress in tangible form, the user is able to let go of those feelings both figuratively and literally. While that by itself induces a reduction in stress level, the apparatus and method goes a step further by enabling the user to then dissolve or otherwise destroy the recorded media. This act of destruction and visual observation of same further serves to facilitate the release of that stress through visual confirmation of its disintegration. Moreover, by providing tools and monitoring equipment, the user is able to quantify that relief in terms of lowered physiological parameters such a blood pressure, heart rate, respiratory rate, and brain activity. This, in turn, enables the user to confirm transformation of his or her nervous system from the predominate use of the sympathetic nervous system to the predominate use of the parasympathetic nervous system.

What is claimed is:

1. A kit adapted to relieve user stress, comprising:
a supply of substrates;
a vessel adapted to hold a supply of solvent; and
a writing instrument adapted to write on the supply of substrates.

2. The kit of claim 1, wherein the supply of substrates are surfactant-based.

3. The kit of claim 2, wherein the writing instrument dispenses a water-based ink.

4. The kit of claim 1, further including a primary lid sized to temporarily close the vessel.

5. The kit of claim 4, wherein the primary lid defines a recess adapted to house the supply of substrates and writing instrument.

6. The kid of claim 5, further including a secondary lid sized to enclose the recess.

7. The kit of claim 1, further including a medical device adapted to measure at least one physiological parameter related to stress level.

8. The kit of claim 7, wherein the medical device monitors a physiological parameter selected from the group consisting of blood pressure, heart rate, respiratory rate, brain function, and combinations thereof.

9. The kit of claim 1, further including a timing device adapted to measure dissolution duration.

10. A method of improving at least one physiological parameter of a user, comprising:
recording information on a tangible substrate;
dissolving the substrate in a solvent; and
allowing the user to observe the substrate transforming from a solid to a liquid for a timed duration sufficient to alter the at least one physiological parameter.

11. The method of claim 9, wherein the recording involves using a writing instrument on the substrate.

12. The method of claim 9, wherein the substrate is surfactant-based and the dissolving involves depositing the substrate in a water-based solvent.

13. The method of claim 9, further including monitoring the at least one physiological parameter with a medical device.

14. The method of claim 12, wherein the monitoring involves monitoring a physiological parameter selected from the group of parameters consisting of blood pressure, heart rate, respiratory rate, brain function, and combinations thereof.

15. The method of claim 9, wherein the allowing alters the at least one physiological parameter downwardly.

16. The method of claim 9, wherein the recording involves information related to a troubling personal thought.

17. A kit adapted to relieve user stress, comprising:
a dissolvable recording media; and
a vessel adapted to hold a solvent.

18. The kit of claim 17, wherein the recording media is surfactant-based and the solvent is water-based.

19. The kit of claim 17, further including a lid adapted to close the vessel, the lid further including an opening adapted to house the dissolvable recording media and a recording instrument.

20. The kit of claim 17, further including a medical device adapted to measure at least one physiological parameter of the user.
